(19) United States
(12) Patent Application Publication
(10) Pub. No.: US 2014/0094748 A1
(43) Pub. Date: Apr. 3, 2014

(54) APPARATUS EXTRACTING MOTHER'S MILK

(71) Applicants: Sang Hyun Hong, Daejeon-si (KR); Jeong Sik Yoo, Chungcheongnam-do (KR); Jung Hyun Hong, Gyeonggi-do (KR)

(72) Inventors: Sang Hyun Hong, Daejeon-si (KR); Jeong Sik Yoo, Chungcheongnam-do (KR); Jung Hyun Hong, Gyeonggi-do (KR)

(21) Appl. No.: 14/118,054

(22) PCT Filed: May 11, 2012
(86) PCT No.: PCT/KR2012/003692

§ 371 Date: Nov. 15, 2013

(30) Foreign Application Priority Data
May 16, 2011 (KR) 10-2011-0045847
Dec. 15, 2011 (KR) 10-2011-0135568

(51) Int. Cl. A61M 1/06

(52) U.S. Cl. CPC A61M 1/06
USPC 604/74

(57) ABSTRACT

There is provided an apparatus extracting mother's milk. More detailedly, the apparatus extracting mothers' milk is provided in order that not only may reverse of mother's milk extracted during extracting mother's milk through an extract apparatus toward an Extract Hose at a pressrue generator be completely discluded by a Pumping Film for preventing reverse, but also repetitive pumpings of a Pumping Film for preventing reverse may be easily carried out.

### TECHNICAL FIELD

The present disclosure relates to an apparatus extracting mother's milk. More specifically, the present disclosure relates to the apparatus extracting mother's milk characterized in that not only may reverse of mother's milk extracted during extracting mother's milk through an extract apparatus toward an Extract Hose at a pressrue generator be completely blocked by a Pumping Film for preventing reverse, but also repetitive pumpings of a Pumping Film for preventing reverse may be easily performed.

### BACKGROUND ART

Generally, an apparatus extracting mother's milk is classified into a functional feeding product capable of completely squeezing mother's milk remaining in mother's breast, when a mother suffers from mastitis as the milk excessively remains in breast or an infant refuses to eat mother's milk, after feeding, and there are types of manual and electric regarding the extract apparatus.

The manual apparatus extracting mother's milk comprises an Absorber contactually covering breast and a storage container (a baby bottle) rendering mother's milk inpoured by an extract of a Vacuum Pumping Device generating extract force with repetitive actions of pressing and unfolding by hand in order to generate air pressure at the Absorber, there was a problem of a low extract efficiency and lots of user's energy cost since air pressure is generated by user's hand, and thus intense air pressure could not be generated.

Recently, an electric apparatus extracting mother's milk is more favored, these apparatuses generally consist of Pressure Generator which periodically generates extract force only or periodically generates extract force and emission force alternately, an Absorber connected to the Pressure Generator and covering breast of mother, and a storage container (a baby bottle) storing milk inpoured through the Absorber.

However, the conventional apparatus for preventing mother's milk from being reversed comprises an Absorber at a side of an Extract Container of an apparatus extracting mother's milk combined at an upper portion of a storage container, a hose connector connected of an Extract Hose of Pressure Generator at another side, and a diaphragm at the Extract Container in order that a reverse of the mother's milk to a direction of the Extract Hose is prevented while delivering extract force to the Absorber as well, there were problems that not only would efficiency of a pump be decreased but also the conventional apparatus would be unsanitary, as partial mother's milk to be dropped to the storage container reverses to the Extract Hose through a bottom portion of the diaphragm mounted at the Extract Container and mother's milk inpours to the inside of the Pressure Generator.

Further, when the Pressure Generator and the Absorber are divided through a device for preventing reverse in order to prevent the reverse, there were problems that not only would extract force generated at the Pressure Generator not be delivered to the Absorber enough, but also continuous repetitive pumpings would be hindered due to an intensified imbalance of pressure around the device for preventing reverse.

### DISCLOSURE

### Technical Problem

The technical problem to be solved by the present disclosure is to provide an apparatus extracting mother's milk which renders milk to be stored to the storage container through an Absorber by inpouring through the Absorber, by rendering extract action at a Pressure Generator delivered to the Absorber enough through the device for preventing reverse.

Further, another technical problem to be solved by the present disclosure is to provide an apparatus extracting mother's milk comprising a device for preventing reverse which can completely block partial extracted milk to be reversed to the Extract Hose connected to the Pressure Generator.

Additionally, another technical problem to be solved by the present disclosure is to provide an apparatus extracting mother's milk having a function of preventing reverse and also rendering extract force generated at Pressure Generator to be delivered to an Absorber enough, and rendering continuous repetitive pumpings performed easily as well.

### Technical Solution

A Pumping Device for an apparatus extracting mother's milk according to an aspect of the present disclosure comprises:
a Pressure Generator generating necessary pressure for extracting mother's milk;
a Pumping Chamber having upper, bottom cases formed as combined, and delivering extract force generated at the Pressure Generator to outside to be used for extracting milk;
a Pumping Film for preventing reverse located in the Pumping Chamber, compressed-fixed toward an inside of a connector of the upper, bottom cases with a certain length, and rendering the Pumping Chamber divided as above and below; and
an Extract Hose delivering the pressure generated at the Pressure Generator to a Pumping Chamber,
wherein the Pumping Chamber emits partial air inside the upper Pumping Chamber to outside through the connector of the upper, bottom cases, when the pressure inside the upper Pumping Chamber formed by the upper case and the Pumping Film for preventing reverse exceeds marginal value.

Hereat, the upper case is funneled of an Extract Hose connecting with Pressure Generator, with a hose connector configured at the upper portion, and the bottom case includes a Rib protruding as a circle at a surface toward an inside of outer peripheral, and it is funneled with a path of extract tube for air, and the Pumping Film for preventing reverse may include a groove formed correspondingly to the Rib of the bottom case at the outer peripheral of the bottom surface.

Further, the Pumping Film for preventing reverse has a thickness of 0.1 to 1.5 mm where the upper, bottom cases are compressed by combining.

Further, the internal pressure has a marginal value of 0.1 mmHg.

Further, a Hook forming two or more protruding portions may be included at the outer peripheral surface of the bottom case, and a Wing portion formed of a Hook hole may be included at the upper case correspondingly to the Hook.

Hereat, the Hook of the bottom case may be formed as dented in comparison with the outer peripheral surface of the bottom case.

An apparatus extracting mother's milk according to another aspect of the present disclosure comprises:
an Extract Container rendering extracted mother's milk to be stored with combined to an upper portion of the storage container;
an Absorber rendering mother's milk to be extracted with funneled to a path of an extract tube for mother's milk formed at a side of the Extract Container and with contacted to breast of mother;
a Pressure Generator generating extract force;
a Pumping Chamber having upper, bottom cases as combined, and delivering extract force generated at the Pressure Generator to a path of an extract tube for air to be used for extracting mother's milk; and
a Pumping Film for preventing reverse located in the Pumping Chamber, compressed-fixed toward the inside of the connector of the upper, bottom cases with a certain length, and rendering the Pumping Chamber divided as above and below;
wherein the Pumping Chamber emits partial air inside the upper Pumping Chamber to outside through the connector of the upper, bottom cases when the pressure inside the upper Pumping Chamber formed by the upper case and the Pumping Film for preventing reverse exceeds marginal value.

Hereat, the upper case is funneled with an Extract Hose connecting with Pressure Generator, with a hose connector comprised at the upper portion, and the bottom case includes a Rib protruding as a circle at a surface toward the inside outer peripheral, and it is funneled with a path of extract tube for air with an extract portion for air formed, and the Pumping Film for preventing reverse may include a groove formed correspondingly to the Rib of the bottom case at the outer peripheral of the bottom surface, of which a thickness ranges from 0.1 to 1.5 mm where upper, bottom cases are compressed by combining.

Additionally, the internal pressure has a marginal value of 0.1 mmHg.

Hereat, the Pumping Chamber may be configured as integrally connected with a path of an extract tube for air of the Extract Container.

### Effect

The apparatus extracting mother's milk according to the present disclosure provides an effect that mother's milk extracted through the device for preventing reverse would not be reversed completely to the Extract Hose by comprising the Pumping Chamber having the device for preventing reverse at the upper portion of the Extract Container connected of the Extract Hose of the Pressure Generator.

Further, it provides an effect that mother's milk is stored to the storage container by delivering an extract act at the Pressure Generator to the device for preventing reverse and thereby causing mother's milk to be extracted due to an extract action obtained from the device for preventing reverse.

In addition, it provides effective functions for preventing reverse and delivering extract force generated at the Pressure Generator to the Absorber enough and further, easily performing continuous repetitive pumpings.

### DESCRIPTION OF DRAWINGS

FIG. 1 illustrates a perspective view of an example of a whole structure of an apparatus extracting mother's milk according to an embodiment of the present disclosure;

FIG. 2 illustrates a partial magnified cross-sectional view of an example of a condition that a Pumping Film for preventing reverse is mounted at a Pumping Chamber of the apparatus extracting mother's milk according to an embodiment of the present disclosure;

FIG. 3 illustrates an applicable cross-sectional view of an example of a condition that a pumping surface of the Pumping Film for preventing reverse mounted at the Pumping Chamber of the apparatus extracting mother's milk according to an exemplary embodiment of the present disclosure pumps upwardly;

FIG. 4 illustrates a perspective view of an example of a Pumping Chamber of an apparatus extracting mother's milk according to an embodiment of the present disclosure;

FIG. 5 illustrates a disassembled perspective view of an example of a condition that a Pumping Film for preventing reverse is mounted at a Pumping Chamber of an apparatus extracting mother's milk according to an embodiment of the present disclosure;

FIG. 6 illustrates a cross-sectional example view of a condition that a Pumping Film for preventing reverse is integrally mounted at a Pumping Chamber of an Extract Container of an apparatus extracting mother's milk according to another embodiment of the present disclosure;

FIG. 7 illustrates a partial magnified cross-sectional view of an example of a condition that a Pumping Film for preventing reverse is mounted at a Pumping Chamber from a structure of the FIG. 6;

FIG. 8 illustrates a perspective view of an example of a whole structure of a Pumping Device for an apparatus extracting mother's milk according to another embodiment of the present disclosure;

FIG. 9 illustrates a partial magnified cross-sectional view of an example of a condition that a Pumping Film for preventing reverse is mounted at a Pumping Chamber of a Pumping Device for an apparatus extracting mother's milk according to another embodiment of the present disclosure;

FIG. 10 illustrates a perspective view of an example of a Pumping Chamber of an apparatus extracting mother's milk according to another embodiment of the present disclosure;

FIG. 11 illustrates a perspective view of an example of a whole structure of an apparatus extracting mother's milk according to another embodiment of the present disclosure; and

FIG. 12 illustrates a cross-sectional example view of an example of a condition that a Pumping Film for preventing reverse is integrally mounted at a Pumping Chamber of an apparatus extracting mother's milk according to another embodiment of the present disclosure.

### BEST MODE

Hereinafter, exemplary embodiments of the present disclosure will be described in detail by referring to the following drawings. As for explaining the present disclosure, a detailed description regarding the related well-known functions or the structure would be omitted when the descriptions therefor may hinder an understanding substance of the present disclosure.

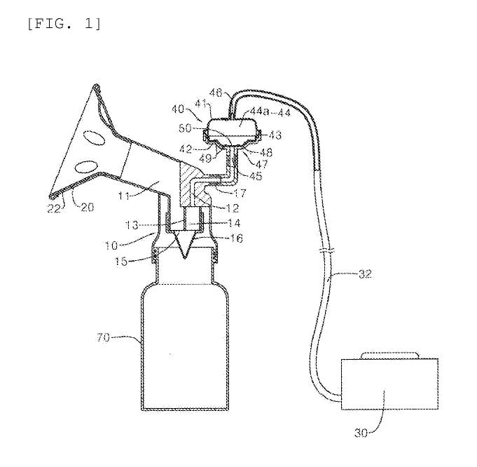

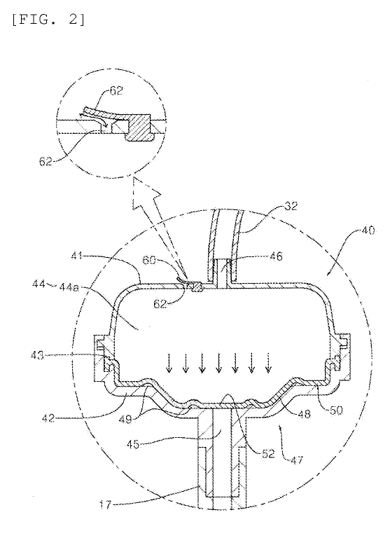

FIG. 1 illustrates a perspective view of an example of a whole structure of an apparatus extracting mother's milk according to an embodiment of the present disclosure.

As illustrated in FIG. 1, the present disclosure provides an apparatus extracting mother's milk comprising:
an Extract Container 10 rendering extracted mother's milk to be stored with combined to an upper portion of the storage container 70;
an Absorber 20 rendering mother's milk to be extracted with funneled to a path of an extract tube for mother's milk 11 formed at a side of the Extract Container 10 and with contacted to breast of mother;
an Extract Hose 32 connecting the Extract Container 10 and a Pressure Generator 30 with connected with a path of an extract tube for air 12 formed at another side of the Extract Container 10; and
a Pressure Generator 30 generating extract force,
wherein a Pumping Chamber 40 is configured as funneled with the path of extract tube for air 12 and mounted with a hose connector 46 at the upper portion,
wherein a Pumping Film for preventing reverse 50 blocks that mother's milk reverses to the Extract Hose 32, and renders pumping action performed with mounted to the inside of the Pumping Chamber 40,
and wherein a Vent Hole 62 comprises a Vacuum Removal Film 60 so that vacuum may be removed by external air's inpouring with a Vent Hole 62 formed at the Pumping Chamber 40.

The Extract Container 10 is shaped of a cylinder having an open bottom portion and it is capable of combining with the storage container 70 storing mother's milk with a connector formed at an outer peripheral portion of a bottom portion. A side of an upper portion of the Extract Container 10 is formed of a path of an extract tube for mother's milk 11 so that a funnel shaped Absorber 20 may be funneled, another side of a path of an extract tube for mother's milk 11 is formed of a path of an extract tube for air 12.

And, the inside of the Extract Container 10 is comprised of a compartment 14 comparting the path of extract tube for mother's milk 11 and the path of extract tube for air 12 into two with a diaphragm 13, and it is formed of a valve for preventing reverse 16 preventing mother's milk in the storage container 70 from being reversed to outside with inserted-combined to an outlet of mother's milk 15 at a bottom portion of the compartment 14, and the valve for preventing reverse 16 is made with a material such as silicon featuring an excellent elasticity and flexibility and the valve is shaped of which an upper portion is wider than a bottom portion.

The Absorber 20 contacts breast of mother with inserted-combined, in order that a contact member 22 made with a material featuring an excellent elasticity and flexibility, may be easily detached, and mother's milk extracted by extract force is stored to the storage container 70 through the outlet of mother's milk 15 via the path of extract tube for mother's milk 11.

The Pressure Generator 30 used in the apparatus extracting mother's milk of the present disclosure may be controllable for pumping force, and it may use a usual draft pump (not illustrated) with regard to generating extract force and extrusion force simultaneously by a linear reciprocating motion of a usual extract pump only generating extract force (not illustrated) or a cylinder, regardless of the capacity of draft pressure of draft pump, this characteristic is accomplished by the Vacuum Removal Film 60 to be described.

FIG. 2 illustrates a partial magnified cross-sectional view of an example of a condition that a Pumping Film for preventing reverse is mounted at a Pumping Chamber of the apparatus extracting mother's milk according to an embodiment of the present disclosure.

As illustrated in FIG. 2, the Pumping Chamber 40 is comprised of the Pumping Chamber 40 inside with the upper, bottom cases 41, 42 combined, a combination end 43 combining the upper, bottom cases 41, 42 comprises an outer peripheral surface of the Pumping Film for preventing reverse 50 with compressed-fixed, and the Pumping Film for preventing reverse 50 divides the Pumping Chamber 40 as above and below, and the bottom Pumping Chamber 44b is formed of the extract portion for air 45 and funneled with the path of extract tube for air 12, and the upper Pumping Chamber 44a is formed of the hose connector 46 and connected to be funneled with the Extract Hose 32, therein.

Further, a bottom surface 47 of the bottom Pumping Chamber 44b configured at the bottom case 42 is formed of a step shaped contact end 48 in which a width of the inside is narrow and a width of the outside is wide, and therein the Pumping Film for reverse 50 becomes contacted by forming an contact protruding portion 49 at each contact end 48, and mother's milk does not remain at the bottom surface 47 in such a way that mother's milk emits downwardly through the extract portion for air 45 of the bottom surface 47 so that mother's milk inpours to the Extract Container 10 with an outlet groove of mother's milk 49a formed at certain interval of the contact protruding portion 49.

The Pumping Film for preventing reverse 50 consists of a material such as silicon featuring an excellent elasticity and flexibility, and it is comprised of a step shaped pumping surface 52 in which an upper portion is wider than a bottom portion, and whose formation is identical to a step shaped contact end 48 formed at the bottom surface 47 of the bottom Pumping Chamber 44b, therein a pumping action performs in that a pumping surface of the Pumping Film for preventing reverse 50 pumps upwardly due to the Pressure Generator 30, during an extract act of the upper Pumping Chamber 44a, and a pumping surface 52 of the Pumping Film for preventing reverse 50 restores as downwardly when a vacuum is removed.

The Vacuum Removal Film 60 is comprised of a material such as silicon featuring an excellent elasticity and flexibility and renders the Vent Hole 62 performing an open and shut action by the Vacuum Removal Film 60 with fixed to a side of the Vent Hole 62 formed at the upper case 41 comprising the upper Pumping Chamber 44a, therein, the Vacuum Removal Film 60 blocks the Vent Hole 62 by being contacted to the Vent Hole 62 when extract force generates at the upper Pumping Chamber 44a, and a pumping surface 52 restores as pumped downwardly in such a way that air may inpour through the Vent Hole 62 by opening the Vent Hole 62, in such a way that the Vacuum Removal Film 60 isolates at the Vent Hole 62 when the extract force of the Pumping Chamber 44 is removed.

In addition, the Vacuum Removal Film 60 performs a function that the outlet pressure may not be delivered excessively at the upper Pumping Chamber 44a since the Vent Hole 62 is open due to the fact that the Vacuum Removal Film 60 blocking the Vent Hole 62 is isolated, when emission force is delivered to the upper Pumping Chamber 44a in the case that the Pressure Generator 30 comprised of the draft pump is used, therefore, the Pressure Generator 30 having a large capacity of the emission pressure of the draft pump may be used.

In the meantime, the Pumping Chamber 40 of the present disclosure may be configured as integrally connected with the path of extract tube for air 12, and may be detachably configured by inserting and connecting to an insert-connect portion 17 of the path of extract tube for air 12 configured at the usual Extract Container 10. As to the detachable type Pumping Chamber 40, the extract portion for air 45 of the bottom Pumping Chamber 44b may be inserted to the insert-connect portion 17.

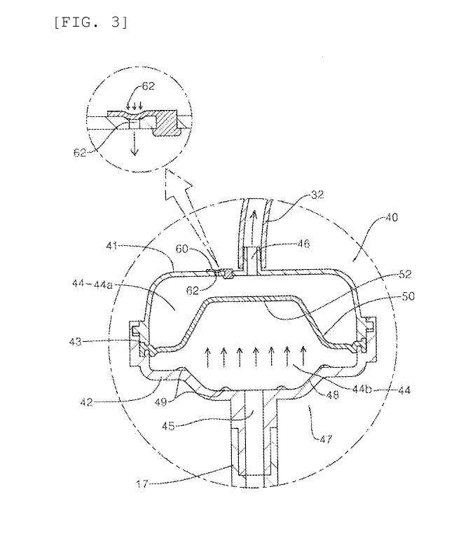

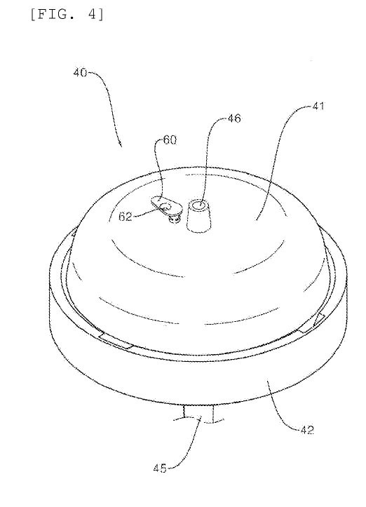

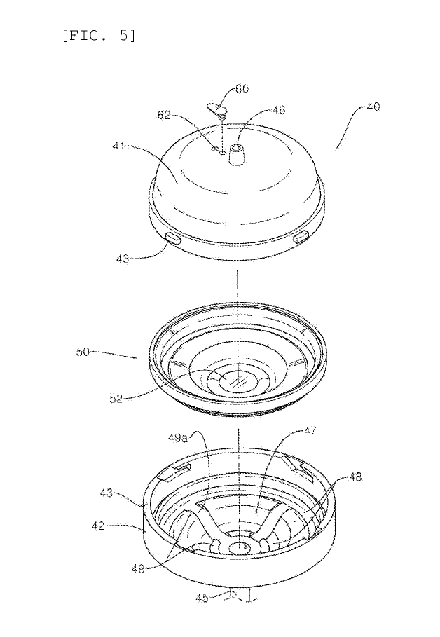

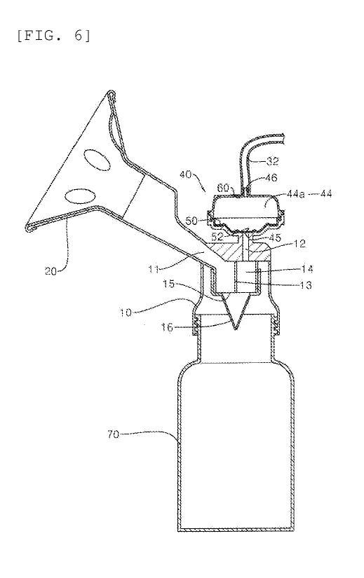

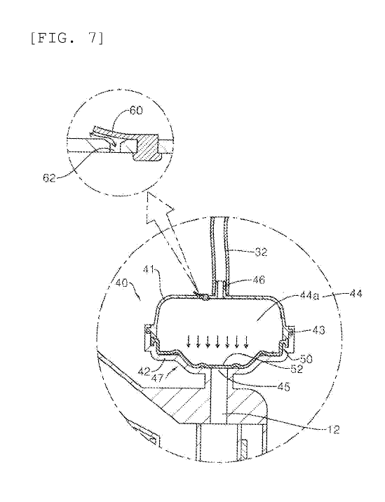

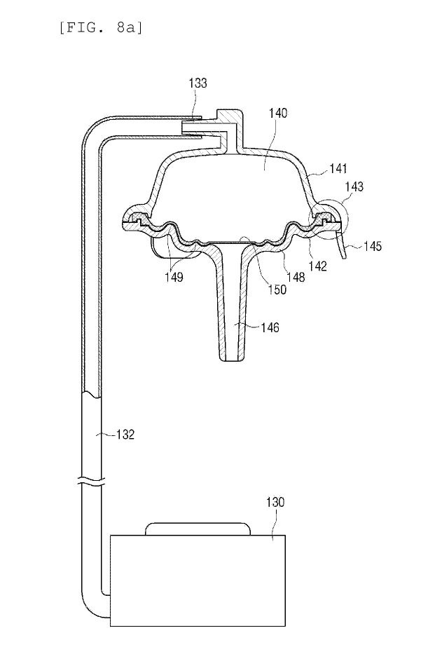

The embodiments according to the apparatus extracting mother's milk configured as above will be detailedly described as below by referring to the following FIGS. 2 to 7.

After combining the storage container 70 to the bottom portion of the Extract Container 10 of the present disclosure, extract force is delivered to the Pumping Chamber 40 and the upper Pumping Chamber 44a funneled with the Extract Hose 32, when the funnel shaped Absorber 20 contacts breast of mother and extract force generates by operating the Pressure Generator 30, whereby the Pumping Film for preventing reverse 50 in which the pumping surface 52 heads to a direction of the bottom surface 47 of the bottom Pumping Chamber 44b renders the pumping surface 52 to be pumping to a direction of the upper Pumping Chamber 44a by an extract action, whereby extract force generates at the path of extract tube for air 12 of the Extract Container 10, whereby mother's milk is extracted through the path of extract tube for mother's milk 11, whereby mother's milk is dropped at the bottom portion of the diaphragm 13, and whereby mother's milk is stored to the storage container 70 via the outlet of mother's milk 15.

At this time, the path of extract tube for mother's milk 11 and the path for extract tube for air 12 basically block an inpour of mother's milk to the Extract Hose 32 of the Pressure Generator 30, in such a blocking function that mothers' milk may not migrate further by the Pumping Film for preventing reverse 50 since the upper side of the path of extract tube for air 12 according to the present disclosure comprises the Pumping Film for preventing reverse 50; whereas, partial mother's milk migrates to the upper side of the path of extract tube for air 12 along with air by extract force of the path of extract tube for air 12 when extract force is delivered through the bottom portion of the diaphragm 13.

That is, the apparatus of the present disclosure provides an extract action of mother's milk by generating extract force of the path of extract tube for air 12 by the Pumping Film for preventing reverse 50 extracted by extract force of the Pressure Generator 30, and the Pumping Film for preventing reverse 50 is very sanitary and it does not hinder durability of the pump, by completely blocking the extract of mother's milk to be inpoured to the inside of the Pressure Generator 30 by mother's milk reversed through the path of extract tube for air 12 since the Pumping Film for preventing reverse 50 is not funneled with the path of extract tube for air 12 and the Extract Hose 32.

In addition, when extract force generates by the upper Pumping Chamber 44a, the Vacuum Removal Film 60 may become transmissive for extract force of the Pressure Generator 30 without influencing extract force of the upper Pumping Chamber 44a since the Vacuum Removal Film 60 blocks the Vent Hole 62 as extracted.

Subsequently, the pumping action may perform in that the pumping surface 52 of the Pumping Film for preventing reverse 50 is restored as pumped downwardly since the Vacuum Removal Film 60 blocking the Vent Hole 62 is isolated from contact condition, and thus the vacuum of the upper Pumping Chamber 44a is removed as external air inpours to the upper Pumping Chamber 44a, when extract force at the upper Pumping Chamber 44a is removed for the extract force at the Pressure Generator 30 is not delivered, whereby mother's milk can be stored to the storage container 70 as the Pumping Film for preventing reverse 50 repetitively performs the pumping action of the pumping surface 52 in that the Pumping Film for preventing reverse 50 pumps upwardly and downwardly.

Further, in the present disclosure, the pumping action may perform in that the pumping surface 52 of the Pumping Film for preventing reverse 50 is compulsively lowered downwardly by delivering emission force to the upper Pumping Chamber 44a with using the Pressure Generator 30 comprising the draft pump drafting a cylinder as a linear motion, accordingly, the extract pump of draft pump may be generally used in the apparatus extracting mother's milk of the present disclosure regardless of the capacity, since the pumping action of the Pumping Film for preventing reverse 50 can be smoothly operated without being influenced to emission force of the Pressure Generator 30, for the Vacuum Removal Film 60 is isolated at the Vent Hole 62 and thereby emitted to outside through the Vent Hole 62 to be open, when the excessive emission pressure inpours at the upper Pumping Chamber 44a in the case that a large capacity of the draft pressure having a large capacity in the draft pump is used.

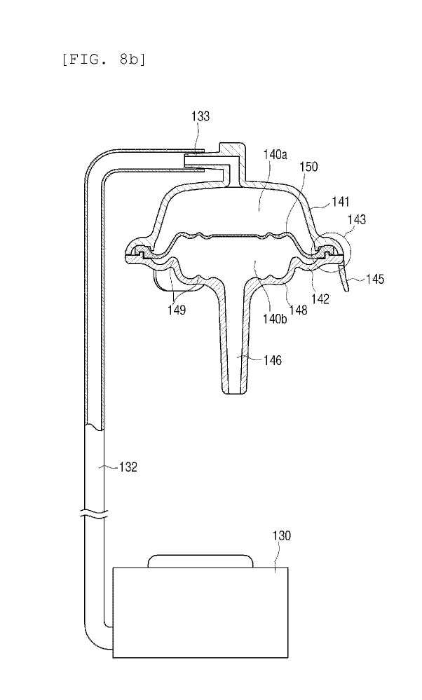

FIG. 8 illustrates a perspective view of an example of a whole structure of a Pumping Device for an apparatus extracting mother's milk according to another embodiment of the present disclosure.

As illustrated in FIG. 8, the Pumping Device for the apparatus extracting mother's milk according to another embodiment of the present disclosure comprises:
a Pressure Generator 130 generating necessary force for extracting mother's milk;
a Pumping Chamber 140 formed of upper, bottom cases 141, 142 with combined, and delivering extract force generated at the Pressure Generator 130 to be used for extracting mother's milk;
a Pumping Film for preventing reverse 150 located in the Pumping Chamber 140, compressed-fixed between the upper, bottom cases 141, 142 toward the inside of the connector 143 of the upper, bottom cases 141, 142 with a certain length, and rendering the Pumping Chamber 140 divided as above and below; and
an Extract Hose 132 delivering the pressure generated at the Pressure Generator 130 to the Pumping Chamber 140,
wherein the Pumping Chamber 140 emits partial air inside the upper Pumping Chamber 140a to outside through the connector 143 of the upper, bottom cases 141, 142 when the pressure inside the upper Pumping Chamber 140a formed by the upper case 141 and the Pumping Film for preventing reverse 150 exceeds marginal value.

As illustrated in FIG. 8b, the Pumping Device for the apparatus extracting mother's milk according to an embodiment of the present disclosure is characterized in that a Pumping Film for preventing reverse 150 pumps upwardly performed by extract force generated by the Pressure Generator 130, and that whereby, the pressure of the bottom Pumping Chamber 140b is lowered, and that whereby, necessary extract force for extracting mother's milk is delivered to outside. Hereat, as to generating necessary pressure for extracting mother's milk, the Pressure Generator 130 may periodically generate extract force only and may periodically generate extract force and emission force alternately, in order for repetitive pumpings of the Pumping Film for preventing reverse 150 to be easy. A skilled person in the art may understand that the Pressure Generator 130 can be realized as an altered formation within the scope of the substance of the present disclosure.

The extract force generated at the Pressure Generator 130 is delivered to the inside of the Pumping Chamber 140 through the Extract Hose 132 connecting the Pressure Generator 130 and the Pumping Chamber 140. Due to the extract force delivered to the inside of the Pumping Chamber 140, the upper, bottom cases 141, 142 are located at the inside of the Pumping Chamber 140 formed by the upper, bottom cases 141, 142 being connected, and the Pumping Film for preventing reverse 150 compressed-fixed between the upper, bottom cases 141, 142 pumps upwardly toward the inside of the connector 143 of the upper, bottom cases 141, 142, with a certain length.

Afterward, if generation of extract force is removed at the Pressure Generator 130 or emission force is generated alternately to extract force, the Pumping Film for preventing reverse 150 pumps downwardly, hereat, the pressure inside the upper Pumping Chamber 140a increases. When the pressure inside the Pumping Chamber 140a increases as above and thereby exceeds a certain marginal value, the continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The Pumping Device for the apparatus extracting mother's milk according to an exemplary embodiment of the present disclosure is characterized in that the partial air inside the upper Pumping Chamber 140a is emitted to outside with a space formed at the connector 143 of the upper, bottom cases 141, 142 when the pressure inside the upper Pumping Chamber 140a exceeds marginal value. As the partial air inside the upper chamber 140a is emitted to outside, the pressure inside the upper Pumping Chamber 140a is maintained constantly. Due to this maintenance, the repetitive pumpings may be feasible as the Pumping Film for preventing reverse 150 may pump upwardly with a same height repetitively.

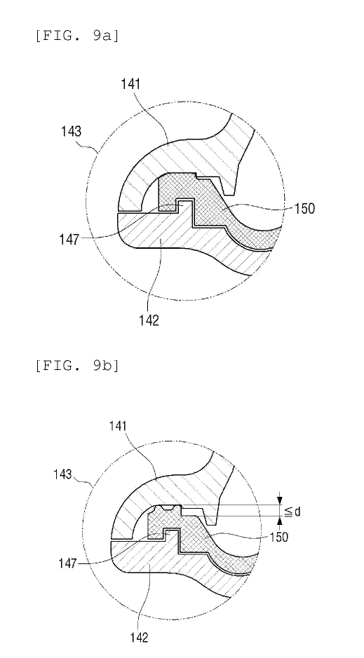

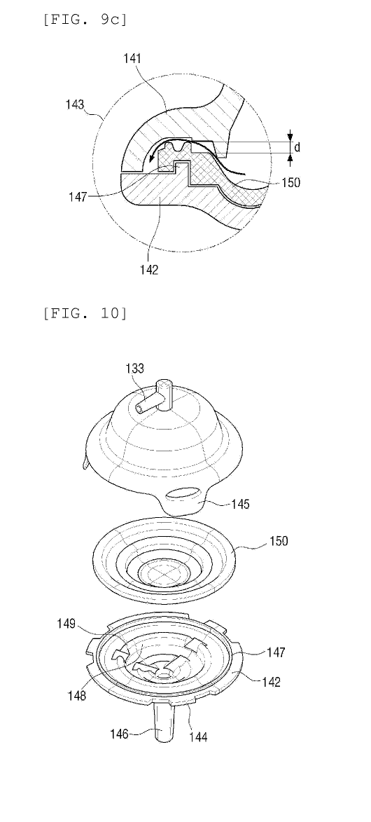

Hereinafter, the structure and function of the apparatus extracting mother's milk according to an exemplary embodiment of the present disclosure will be detailedly described by referring to FIGS. 9 and 10.

FIG. 9 illustrates a partial magnified cross-sectional view of an example of a condition that a Pumping Film for preventing reverse 150 is mounted at a Pumping Chamber 140 of a Pumping Device for an apparatus extracting mother's milk according to an embodiment of the present disclosure; and

FIG. 10 illustrates a perspective view of an example of a Pumping Chamber 140 of a Pumping Device for an apparatus extracting mother's milk according to an embodiment of the present disclosure.

As illustrated in FIGS. 9 and 10, as for the Pumping Device for the apparatus extracting mother's milk according to an embodiment of the present disclosure, the Pumping Chamber 140 consists of the upper case 141, the Pumping Film for preventing reverse 150 and the bottom case 142. The upper case 141 is comprised of the hose connector 133 at the upper portion and it is funneled of the Extract Hose 132 connecting the Pressure Generator 130. The bottom case 142 includes a protruding Rib 147 as a circle at a surface toward the inside of the outer peripheral, therein, the extract portion for air 146 is formed at the bottom, and the bottom portion is funneled with the path of extract tube for air. The Pumping Film for preventing reverse 150 includes a groove formed correspondingly to the Rib 147 of the bottom case 142 at the outer peripheral surface of the bottom portion, and it is compressed-fixed between the upper, bottom cases 141, 142 toward the inside of the connector 143 of the upper, bottom cases 141, 142.

As illustrated in FIG. 9a, the Pumping Film for preventing reverse 150 is compressed-fixed between the connectors 143 of the upper, bottom cases 141, 142. The Pumping Film for preventing reverse 150 performs a role as a ceiling member by being compressed between the upper case 141 and the bottom case 142.

After the generation of the Pressure Generator 130, the Pumping Film for preventing reverse 150 pumps downwardly, of which pumped upwardly, when the generation of extract force is removed at the Pressure Generator 130 or emission force is generated alternately to the extract force. Due to this downward pumping, the pressure inside the upper Pumping Chamber 140a increases. That is, as illustrated in FIG. 9b, the upper case 141 and the bottom case 142 are isolated as the pressure inside the upper Pumping Chamber 140a increases. At this time, the Pumping Film for preventing reverse 150 performs a role as a ceiling member preventing the air inside the Pumping Chamber 140 from being emitted to outside, by ceiling the isolated space by the Pumping Film for preventing reverse 150 compressed-fixed between the upper, bottom cases 141, 142.

However, as illustrated in FIG. 9c, when the pressure inside the upper Pumping Chamber 140a exceeds marginal value, a space is made between the upper case 141 and the Pumping Film for preventing reverse 150, the partial air inside the upper Pumping Chamber 140a is emitted to outside through this space. Due to this emission, the pressure inside the upper Pumping Chamber 140a is lowered as equal to or less than the marginal value again. As described above, magnitude of the space that the internal air emits to outside is determined by a level that the Pumping Film for preventing reverse 150 compressed-fixed between the upper, bottom cases 141, 142 covers the formed space by the pressure difference, and therefore, the amount of air emitted to outside is controlled by this determination.

In an embodiment applicable to the present disclosure, a portion that the Pumping Film for preventing reverse 150 is compressed-fixed between the connectors 143 of the upper, bottom cases 141, 142 may be formed as a circle. At this time, in an exemplary embodiment of the present disclosure, the portion compressed-fixed may be formed as two circles. A skilled person in the art may understand that the portion compressed-fixed can be realized as an altered formation within the scope of the substance of the present disclosure.

The marginal value of the pressure inside the upper Pumping Chamber 140a according to an exemplary embodiment of the present disclosure is 0.1 mmHg. The Pumping Film for preventing reverse 150 pumps downwardly and the pressure inside the upper Pumping Chamber 140a is increased, when the generation of extract force is removed at the Pressure Generator 130 or emission force is generated alternately to the extract force. Hereat, the internal pressure exceeds 0.1 mmHg, a space is formed at the connector 143 as the upper, bottom cases 141, 142 are isolated each other and thus, the partial air inside the upper Pumping Chamber 140a is emitted to outside trough the space formed. Due to the emission of the partial air inside the upper Pumping Chamber 140a, the pressure inside the upper Pumping Chamber 140a is lowered as equal to or less than 0.1 mmHg. If a condition that the pressure inside the upper Pumping Chamber 140a exceeds 0.1 mmHg is maintained, the continuous repetitive pumpings may be hindered since the extract force may not be delivered enough to the Pumping Film for preventing reverse 150 even if a next extract force is generated at the Pressure Generator 130. Further, the excessive pressure stacked at the Pumping Chamber 140 may cause the case to be cracked or exploded.

Additionally, the Pumping Film for preventing reverse 150 of the Pumping Chamber 140 according to an exemplary embodiment of the present disclosure is characterized in that the Pumping Film for preventing reverse 150 has a thickness (d) of 0.1 to 1.5 mm where the upper, bottom cases 141, 142 are compressed by combining. When the thickness compressed becomes equal to or less than 0.1 mm, the Pumping Film for preventing reverse cannot do a role as a ceiling member. More detailedly, when the extract action operates by the Pressure Generator 130, the extract force generated at the Pressure Generator 130 would not be delivered to the Pumping Film for preventing reverse 150 enough since the external air inpours to the inside of the Pumping Chamber 140 through a crack at the connector 143 of the upper, bottom cases 141, 142 in the case that the thickness compressed becomes equal to or less than 0.1 mm. Hence, continuous repetitive pumping actions may not be performed smoothly. Therefore, the Pumping Device cannot perform normally the action of extracting mother's milk when the thickness that the Pumping Film for preventing reverse 150 is compressed becomes equal to or less than 0.1 mm. Further, if the thickness compressed becomes 1.5 mm or more, the pressure inside the upper Pumping Chamber 140a exceeds the marginal value, whereby the upper, bottom cases 141, 142 are isolated each other, and whereby the space would be clogged by the Pumping Film for preventing reverse 150 even if a space is formed in the connector 143. Therefore, the air inside the Pumping Chamber 140 may not be easily emitted to outside due to the Pumping Film for preventing reverse 150, the excessive pressure is implemented to the Pumping Chamber 140, due to this implementation, the continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The bottom portion of the bottom case 142 is formed of the extract portion for air 146 and it is funneled with the path of extract tube for air and it is formed of a step shaped contact end 148 in which the bottom Pumping Chamber 140b configured by the bottom case 142 has a narrow inside and a wide outside in width, whereby the Pumping Film for preventing reverse 150 may be contacted by forming the contact protruding portion 149 protruding at each contact end 148. The Pumping Film for preventing reverse 150 compressed-fixed between the upper, bottom cases 141, 142 prevents the mother's milk from being reversed to the upper case 141 and the Pressure Generator 130 even if mother's milk is reversed through the extract portion for air 146 of the bottom case 142 with the structure described above. In addition, the mother's milk is not remained in the bottom case 142 as the bottom case 142 is formed as a step shaped contact end 148 in which an upper portion is wider than a bottom portion, in order that mother's milk would be emitted downwardly through the extract portion for air 146 of the bottom surface, even if the mother's milk is reversed.

The Pumping Device for the apparatus extracting mother's milk according to an exemplary embodiment of the present disclosure comprises:
a Hook 144 forming two or more protruding portions at the outer peripheral surface of the bottom case 142 of the Pumping Device; and
a Wing portion 145 formed of a Hook hole correspondingly to the Hook 144 at the upper case 141. At this time, the combination between the upper, bottom cases 141, 142 is performed in a so-called lock and lock method that the Hook 144 of the bottom case 142 is fixed by the Hook hole of the Wing portion 145. The Hook 144 of the bottom case 142 may be formed as dented in comparison with the outer peripheral surface of the bottom case. If the Hook 144 of the bottom case 142 is formed as dented in comparison with the outer peripheral surface, efficiency of the extract action for mother's milk may be enhanced by reducing the space generated in the connector 143 of the upper, bottom cases 141, 142, when the pressure inside the upper Pumping Chamber 140a exceeds the marginal value. Hereat, the number of the Hook 144 and Hook hole may be two or more. The number of the Hook 144 and Hook hole may further be three; however, a skilled person in the art may understand that the number of the Hook 144 and Hook hole can be realized as an altered formation within the scope of the substance of the present disclosure.

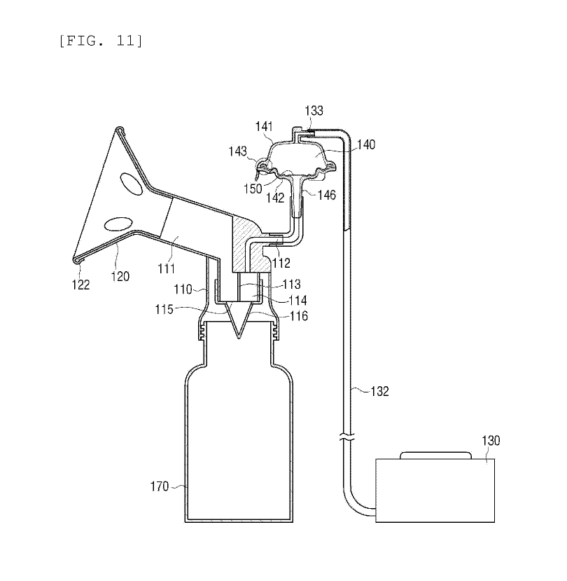

FIG. 11 illustrates a perspective view of an example of a whole structure of an apparatus extracting mother's milk according to an embodiment of the present disclosure.

As illustrated in FIG. 11, the apparatus extracting according to an exemplary embodiment of the present disclosure comprises:
an Extract Container 110 rendering mother's milk extracted with combined to the upper portion of the storage container 170 to be stored;
an Absorber 120 rendering mother's milk to be extracted with funneled with a path of an extract tube for mother's milk 111 formed at a side of the Extract Container 110;
a Pressure Generator 130 generating extract force;
a Pumping Chamber 140 having the upper, bottom cases 141, 142 as combined, and delivering extract force to the path of extract tube for air 112 in order for the extract force generated at the Pressure Generator 130 to be used for extracting mother's milk; and
a Pumping Film for preventing reverse 150 located in the Pumping Chamber 140, compressed-fixed between the upper, bottom cases 141, 142 toward the inside of the connector 143 of the upper, bottom cases 141, 142 with a certain length, and dividing the Pumping Chamber 140 as above and below,
wherein the Pumping Chamber 140 emits partial air inside the upper Pumping Chamber 140a to outside through the connector 143 of the upper, bottom cases 141, 142, when the pressure inside the upper Pumping Chamber 140a formed by the upper case 141 and the Pumping Film for preventing reverse 150 exceeds marginal value.

The Extract Container 10 is shaped of a cylinder having an open bottom portion and it is capable of combining with the storage container 70 storing mother's milk with a connector formed at an outer peripheral portion of a bottom portion, therein, a side of the upper portion of the extract portion 110 is formed of the path of extract tube for mother's milk 111 in order for the funnel shaped Absorber 120 to be funneled, another side of the path of extract tube for mother's milk 111 is formed of the path of extract tube for air 112, therein.

And, the inside of the Extract Container 110 comprises: a compartment 114 comparting the path of extract tube for mother's milk 111 and the path of extract tube for air 112 into two with a diaphragm 113. A valve for preventing reverse 116 preventing mother's milk in the storage container 170 from being reversed to outside with inserted and combined to the outlet of mother's milk 115 at the bottom portion of the compartment 114, wherein the valve for preventing reverse 116 is made with a material such as silicon featuring an excellent elasticity and flexibility and configured as a shape in which an upper portion is wider than a bottom portion.

The Absorber 120 is contacted to breast of mother with inserted and combined in order for the contact member 112 made with a material featuring an excellent elasticity and flexibility to be easily detached, and mother's milk extracted by extract force is stored to the storage container 170 through the outlet of mother's milk 115 via the path of extract tube for mother's milk 111.

An embodiment of the present disclosure indicates that extract force is delivered to the Pumping Chamber 140 and the upper Pumping Chamber 140a funneled with the Extract Hose 132 when extract force generates, by combining the storage container 170 to the bottom portion of the Extract Container 110 and contacting the funnel shaped Absorber 120 to breast of mother and operating the Pressure Generator 130. Due to this delivery, the Pumping Film for preventing reverse 150 in which the pumping surface heads to the direction of bottom surface of bottom Pumping Chamber 140b renders the pumping surface to be pumping to the direction of the upper Pumping Chamber 140a by the extract action, whereby the extract force generates at the path of extract tube for air 112 of the Extract Container 110, whereby mother's milk is extracted through the path of extract tube for mother's milk 111, and whereby mother's milk is dropped to the bottom portion of the diaphragm and stored to the storage container 170 via the outlet of mother's milk 115.

More detailedly, the apparatus extracting mother's milk according to an embodiment of the present disclosure is characterized in that the Pumping Film for preventing reverse 150 is pumped upwardly by the extract force generated by the Pressure Generator 130, and that the extract force necessary for extracting mother's milk is delivered to outside as the pressure of the bottom Pumping Chamber 140b lowers due to the upward pumping. Hereat, as for generating the necessary pressure for extracting mother's milk, the Pressure Generator 130 may periodically generate emission force only, or may periodically generate extract force and emission force alternately in order for the repetitive pumpings of the Pumping Film for preventing reverse 150 to be easy. A skilled person in the art may understand that the Pressure Generator 130 can be realized as an altered formation within the scope of the substance of the present disclosure.

The extract force generated at the Pressure Generator 130 is delivered to the inside of the Pumping Chamber 140 through the Extract Hose 132 connecting the Pressure Generator 130 and the Pumping Chamber 140. Due to the extract force delivered to the inside of the Pumping Chamber 140 as performed above, the upper, bottom cases 141, 142 are located in the Pumping Chamber 140 having the upper, bottom cases 141, 142 as combined. The Pumping Film for preventing reverse 150 compressed-fixed between the upper, bottom cases 141, 142 toward the inside of the connector 143 of the upper, bottom cases 141, 142 may pump upwardly with a certain length.

Afterward, the Pumping Film for preventing reverse 150 pumps downwardly when the generation of the extract force is removed at the Pressure Generator 130 or the emission force is generated alternately to the extract force; hereat, the pressure inside the upper Pumping Chamber 140a increases.

If a condition that the pressure inside the upper Pumping Chamber 140a exceeds a certain marginal value due to the increase of the pressure inside the upper Pumping Chamber 140a is maintained, continuous repetitive pumpings may be hindered as the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The apparatus extracting mother's milk according to an exemplary embodiment of the present disclosure is characterized in that the partial air inside the upper Pumping Chamber 140a is emitted to outside by being formed of a space at the connector 143 of the upper, bottom cases 141, 142 when the pressure inside the upper Pumping Chamber 140a exceeds marginal value. As the partial air inside the upper chamber 140a emits as described above, the pressure inside the upper Pumping Chamber 140a is maintained constantly. According to this constant maintenance, the repetitive pumpings may be feasible as the Pumping Film for preventing reverse 150 may pump upwardly with a same height repetitively.

The upper case 141 is comprised of the hose connector 133 at the upper portion and it is funneled of the Extract Hose 132 connecting the Pressure Generator 130. The bottom case 142 includes a protruding Rib 147 as a circle at a surface toward the inside of the outer peripheral, and the extract portion for air 146 is formed at the bottom portion, and the bottom portion is funneled with the path of extract tube for air 112. The Pumping Film for preventing reverse 150 includes a groove formed correspondingly to the Rib 147 of the bottom case 142 at the outer peripheral of the bottom surface, and it is compressed-fixed between the upper, bottom cases 141, 142 toward the inside of the connector 143 of the upper, bottom cases 141, 142.

The Pumping Film for preventing reverse 150 is compressed-fixed between the upper, bottom cases 141, 142. When the pressure inside the upper Pumping Chamber 140a exceeds the marginal value, the partial air inside the upper Pumping Chamber 140a is emitted to outside through a space formed at the connector 143 of the upper, bottom cases 141, 142. The Pumping Film for preventing reverse 150 performs a function of controlling the amount of air emitted to outside between the upper, bottom cases 141, 142. More detailedly, when the pressure inside the upper Pumping Chamber 140a exceeds the marginal value, a space is generated at the connector 143 as the upper, bottom cases 141, 142 are isolated each other upon the effect above. Due to this space generated, magnitude of the space that air emits to outside is determined by a level that the Pumping Film for preventing reverse 150 compressed-fixed between the upper, bottom cases 141, 142 covers the formed space, and therefore, the amount of air emitted to outside is controlled by this determination.

The marginal value of the pressure inside the upper Pumping Chamber 140a according to an exemplary embodiment of the present disclosure is 0.1 mmHg. The Pumping Film for preventing reverse 150 pumps downwardly and the pressure inside the upper Pumping Chamber 140a is increased, when the generation of extract force at the Pressure Generator 130 is removed or emission force is generated alternately to the extract force. At this time, when the internal pressure exceeds 0.1 mmHg, a space is formed at the connector 143 as the upper, bottom cases 141, 142 are isolated each other and thus, the partial air inside the upper Pumping Chamber 140a is emitted to outside trough the space formed. Due to the emission of the partial air inside the upper Pumping Chamber 140a, the pressure inside the upper Pumping Chamber 140a is lowered as equal to or less than 0.1 mmHg. If the pressure inside the upper Pumping Chamber 140a exceeds 0.1 mmHg, the continuous repetitive pumpings may be hindered since the extract force may not be delivered enough to the Pumping Film for preventing reverse 150 even if a next extract force is generated at the Pressure Generator 130. Further, the excessive pressure stacked at the Pumping Chamber 140 may cause the case to be cracked or exploded.

Additionally, the Pumping Film for preventing reverse 150 of the Pumping Chamber 140 according to an exemplary embodiment of the present disclosure is characterized of having a thickness (d) of 0.1 to 1.5 mm where the upper, bottom cases 141, 142 are compressed by combining. When the thickness compressed becomes equal to or less than 0.1 mm, the Pumping Film for preventing reverse 150 may not do a role as a ceiling member. More detailedly, when the extract motion operates by the Pressure Generator 130, the extract force generated at the Pressure Generator 130 would not be delivered to the Pumping Film for preventing reverse 150 enough since the external air inpours to the inside of the Pumping Chamber 140 through a crack at the connector 143 of the upper, bottom cases 141, 142 if the thickness compressed becomes equal to or less than 0.1 mm. Hence, continuous repetitive pumping actions may not be performed smoothly. Therefore, the Pumping Device cannot perform normally the extract action when the thickness that the Pumping Film for preventing reverse 150 is compressed becomes equal to or less than 0.1 mm. Further, if the thickness compressed becomes 1.5 mm or more, the pressure inside the upper Pumping Chamber 140a exceeds the marginal value, whereby the upper, bottom cases 141, 142 are isolated each other, and whereby the space would be clogged by the Pumping Film for preventing reverse 150 even if a space is formed at the connector 143. Therefore, the air inside the Pumping Chamber 140 may not be easily emitted to outside due to the Pumping Film for preventing reverse 150, the excessive pressure is implemented to the Pumping Chamber 140, due to this implementation, the continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The bottom portion of the bottom case 142 is formed of the extract portion for air 146 and it is funneled with the path of extract tube for air 112 and it is formed of a step shaped contact end 148 in which the bottom Pumping Chamber 140b configured by the bottom case 142 has a narrow inside and a wide outside in width. The Pumping Film for preventing reverse 150 may be contacted by forming the contact protruding portion 149 protruding at each contact end 148. The Pumping Film for preventing reverse 150 compressed-fixed between the upper, bottom cases 141, 142 prevents the mother's milk from being reversed to the upper case 141 and the Pressure Generator 130 even if mother's milk is reversed through the path of extract tube for air 112 of the bottom case 142 with the structure above. In addition, the mother's milk is not remained in the bottom case 142 as the bottom case 142 is formed as a step shaped contact end 148 in which an upper portion is wider than a bottom portion in order that mother's milk would be emitted downwardly through the extract portion for air 146 at the bottom surface, even if the mother's milk is reversed.

The apparatus extracting mother's milk according to an exemplary embodiment of the present disclosure comprises:
a Hook 144 forming two or more protruding portions at the outer peripheral surface of the bottom case 142; and
a Wing portion 145 formed of a Hook hole correspondingly to the storage container 170 at the upper case 141. At this time, the combination between the upper, bottom cases 141, 142 is performed in a so-called lock and lock method that the Hook 144 of the bottom case 142 is fixed by the Hook hole of the Wing portion 145. The Hook 144 of the bottom case 142 may be formed as dented in comparison with the outer peripheral surface of the bottom case. If the Hook 144 of the bottom case 142 is formed as dented in comparison with the outer peripheral surface, efficiency of the extract action for mother's milk may be enhanced by reducing the space generated in the connector 143 of the upper, bottom cases 141, 142, when the pressure inside the upper Pumping Chamber 140a exceeds the marginal value. Hereat, the number of the Hook 144 and Hook hole may be two or more. The number of the Hook 144 and Hook hole may further be three; a skilled person in the art may understand that the number of the Hook 144 and Hook hole can be realized as an altered formation within the scope of the substance of the present disclosure.

At this time, the Pumping Chamber 140 of the apparatus extracting mother's milk is configured detachably in order to be used by inserting and connecting to the insert-connect portion comprised in usual Extract Container 110. That is, it may be used by being combined with an Extract Container 110 of an apparatus extracting mother's milk differently formed with the Extract Container 110 according to an exemplary embodiment of the present disclosure, not because the path of extract tube for air between the Pumping Chamber 140 and the Extract Container 110 is integrated, but it is detachable. A skilled person in the art may understand that the Extract Container 110 of the apparatus extracting mother's milk can be realized as an altered formation within the scope of the substance of the present disclosure.

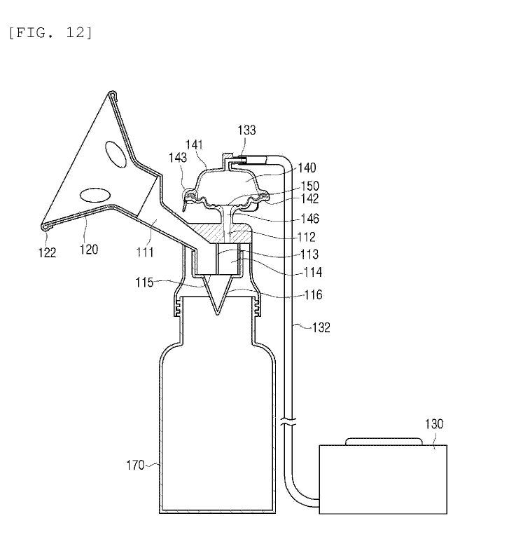

FIG. 12 illustrates a cross-sectional example view of a condition that a Pumping Film for preventing reverse 150 is integrally mounted at a Pumping Chamber 140 of an apparatus extracting mother's milk according to another embodiment of the present disclosure.

As illustrated in FIG. 12, a Pumping Chamber 140 of the apparatus extracting mother's milk according to another embodiment of the present disclosure is integrally connected with the path of extract tube for air 112 of the Extract Container 110.

The apparatus extracting mother's milk according to another aspect of the present disclosure comprises:
an Extract Container 110 rendering mother's milk extracted with combined to the upper portion of the storage container 170 to be stored;
an Absorber 120 rendering mother's milk to be extracted with funneled with a path of an extract tube for mother's milk 111 formed at a side of the Extract Container 110 and with contacted to breast of mother;
an Extract Hose 132 connecting the Extract Container 110 and Pressure Generator 130 with connected with a path of extract tube for air 112 formed at another side of the Extract Container 110;
a Pressure Generator 130 generating extract force;
a Pumping Chamber 140 formed of the upper, bottom cases 141, 142 with combined, and delivering extract force to outside in order for the extract force generated at the Pressure Generator 130 to be used for extracting mother's milk; and
a Pumping Film for preventing reverse 150 located in the Pumping Chamber 140, compressed-fixed between the upper, bottom cases 141, 142 toward the inside of the connector 143 of the upper, bottom cases 141, 142 with a certain length, and dividing the Pumping Chamber 140 as above and below,
wherein the Pumping Chamber 140 emits partial air inside the upper Pumping Chamber 140a to outside through the connector 143 of the upper, bottom cases 141, 142, when the pressure inside the upper Pumping Chamber 140a formed by the upper case 141 and the Pumping Film for preventing reverse 150 exceeds marginal value.

The Extract Container 110 is shaped of a cylinder having the open bottom portion, and the connector is formed at the bottom peripheral in order that the storage container 170 may be combined, a side of the upper portion of the extract portion 110 is formed of the path of extract tube for mother's milk 111 in order for the funnel shaped Absorber 120 to be funneled, therein, and another side of the path of extract tube for mother's milk 111 is formed of the path of extract tube for air 112, therein.

And, the inside of the Extract Container 110 comprises: a compartment 114 comparting the path of extract tube for mother's milk 111 and the path of extract tube for air 112 into two with a diaphragm 113. A valve for preventing reverse 116 preventing mother's milk in the storage container 170 from being reversed to outside with inserted and combined to the outlet of mother's milk 115 at the bottom portion of the compartment 114, wherein the valve for preventing reverse 116 is made with a material such as silicon featuring an excellent elasticity and flexibility and configured as a shape in which an upper portion is wider than a bottom portion.

The Absorber 120 is contacted to breast of mother with inserted and combined in order for the contact member 112 made with a material featuring an excellent elasticity and flexibility to be easily detached, and mother's milk extracted by extract force is stored to the storage container 170 through the outlet of mother's milk 115 via the path of extract tube for mother's milk 111.

An embodiment of the present disclosure indicates that extract force is delivered to the Pumping Chamber 140 and the upper Pumping Chamber 140a funneled with the Extract Hose 132 when extract force is generated, by combining the storage container 170 to the bottom portion of the Extract Container 110 and contacting the funnel shaped Absorber 120 to breast of mother and operating the Pressure Generator 130. Due to this delivery, the Pumping Film for preventing reverse 150 in which the pumping surface heads to the direction of bottom side of bottom Pumping Chamber 140b renders the pumping surface to be pumping to the direction of the upper Pumping Chamber 140a by the extract action, and thus the extract force generates at the path of extract tube for air 112 of the Extract Container 110, and thus mother's milk is extracted through the path of extract tube for mother's milk 111 and thus mother's milk is dropped to the bottom portion of the diaphragm and stored to the storage container 170 via the outlet of mother's milk 115.

More detailedly, the apparatus extracting mother's milk according to an embodiment of the present disclosure is characterized in that the Pumping Film for preventing reverse 150 is pumped upwardly by the extract force generated by the Pressure Generator 130, and that the extract force necessary for extracting mother's milk is delivered to outside as the pressure of the bottom Pumping Chamber 140b lowers due to the upward pumping. Hereat, as for generating the necessary pressure for extracting mother's milk, the Pressure Generator 130 may periodically generate emission force only, or may periodically generate extract force and emission force alternately in order that the repetitive pumpings of the Pumping Film for preventing reverse 150 may be easy. A skilled person in the art may understand that the Pressure Generator 130 can be realized as an altered formation within the scope of the substance of the present disclosure.

The extract force generated at the Pressure Generator 130 is delivered to the inside of the Pumping Chamber 140 through the Extract Hose 132 connecting the Pressure Generator 130 and the Pumping Chamber 140. Due to the extract force delivered to the inside of the Pumping Chamber 140, the Pumping Film for preventing reverse 150 pumps upwardly with a certain length, which is located in the Pumping Chamber 140 formed of the upper, bottom cases 141, 142 with combined, and which is compressed-fixed between the upper, bottom cases 141, 142 toward the inside of the connector 143 of the upper, bottom cases 141, 142.

Afterward, the Pumping Film for preventing reverse 150 pumps downwardly if the generation of the extract force is removed at the Pressure Generator 130 or the emission force is generated alternately to the extract force; hereat, the pressure inside the upper Pumping Chamber 140a increases.

When the pressure inside the upper Pumping Chamber 140a maintains the excess of a certain marginal value due to the increase of the pressure inside the upper Pumping Chamber 140a, continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The apparatus extracting mother's milk according to an exemplary embodiment of the present disclosure is characterized in that the partial air inside the upper Pumping Chamber 140a is emitted to outside by being formed of a space at the connector 143 of the upper, bottom cases 141, 142 when the pressure inside the upper Pumping Chamber 140a exceeds marginal value. As the partial air inside the upper chamber 140a is emitted as described above, the pressure inside the upper Pumping Chamber 140a is maintained constantly. According to this pressure maintained constantly, the repetitive pumpings may be feasible since the Pumping Film for preventing reverse 150 may pump upwardly repetitively with a same height.

The upper case 141 is comprised of the hose connector 133 at the upper portion and it is funneled of the Extract Hose 132 connecting the Pressure Generator 130. The bottom case 142 includes a protruding Rib 147 as a circle at a surface toward the inside of the outer peripheral, therein, the extract portion for air 146 is formed at the bottom, and the bottom is funneled with the path of extract tube for air 112. The Pumping Film for preventing reverse 150 includes a groove formed correspondingly to the Rib 147 of the bottom case 142, at the outer peripheral of the bottom surface and it is compressed-fixed between the upper, bottom cases 141, 142 toward the inside of the connector 143 of the upper, bottom cases 141, 142.

The Pumping Film for preventing reverse 150 is compressed-fixed between the upper, bottom cases 141, 142. When the pressure inside the upper Pumping Chamber 140a exceeds the marginal value, the partial air inside the upper Pumping Chamber 140a is emitted to outside through a space formed at the connector 143 of the upper, bottom cases 141, 142. The Pumping Film for preventing reverse 150 performs a function of controlling the amount of air emitted to outside between the upper, bottom cases 141, 142. More detailedly, when the pressure inside the upper Pumping Chamber 140a exceeds the marginal value, the space is generated at the connector 143 as the upper, bottom cases 141, 142 are isolated each other upon the effect above, due to this space generated, magnitude of the space that air emits to outside is determined by a level that the Pumping Film for preventing reverse 150 compressed-fixed between the upper, bottom cases 141, 142 covers the formed space, and therefore, the amount of air emitted to outside is controlled by this determination.

The marginal value of the pressure inside the upper Pumping Chamber 140a according to an exemplary embodiment of the present disclosure is 0.1 mmHg. The Pumping Film for preventing reverse 150 pumps downwardly and the pressure inside the upper Pumping Chamber 140a increases, when the generation of extract force is removed at the Pressure Generator 130 or emission force is generated alternately to the extract force. At this time, when the internal pressure exceeds 0.1 mmHg, a space is formed at the connector 143 as the upper, bottom cases 141, 142 are isolated each other and thus, the partial air inside the upper Pumping Chamber 140a is emitted to outside trough the space formed. Due to the emission of the partial air inside the upper Pumping Chamber 140a, the pressure inside the upper Pumping Chamber 140a is lowered as equal to or less than 0.1 mmHg. If the pressure inside the upper Pumping Chamber 140a exceeds 0.1 mmHg, the continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

Additionally, the Pumping Film for preventing reverse 150 of the Pumping Chamber 140 according to an exemplary embodiment of the present disclosure is characterized in that the Pumping Film for preventing reverse 150 has a thickness (d) of 0.1 to 1.5 mm where the upper, bottom cases 141, 142 are compressed by combining. When the thickness compressed becomes equal to or less than 0.1 mm, the Pumping Film for preventing reverse 150 may not do a role as a ceiling member. More detailedly, when the extract action operates by the Pressure Generator 130, the extract force generated at the Pressure Generator 130 would not be delivered to the Pumping Film for preventing reverse 150 enough since the external air inpours to the inside of the Pumping Chamber 140 through a crack at the connector 143 of the upper, bottom cases 141, 142 if the thickness compressed becomes equal to or less than 0.1 mm. Hence, continuous repetitive pumping actions may not be performed smoothly. Therefore, the Pumping Device cannot perform normally the extract action when the thickness that the Pumping Film for preventing reverse 150 is compressed becomes equal to or less than 0.1 mm. Further, if the thickness compressed becomes 1.5 mm or more, the pressure inside the upper Pumping Chamber 140a exceeds the marginal value, whereby the upper, bottom cases 141, 142 are isolated each other, and whereby the space would be clogged by the Pumping Film for preventing reverse 150 even if a space is formed at the connector 143. Therefore, the air inside the Pumping Chamber 140 may not be easily emitted to outside due to the Pumping Film for preventing reverse 150, the excessive pressure is implemented to the Pumping Chamber 140, due to this implementation, the continuous repetitive pumpings may be hindered since the extract force may not be delivered to the Pumping Film for preventing reverse 150 enough even if a next extract force is generated at the Pressure Generator 130. Further, the excessive pressure stacked at the Pumping Chamber 140 may cause the case to be cracked or exploded.

The bottom portion of the bottom case 142 is formed of the extract portion for air 146 and thus it is funneled with the path of extract tube for air 112 and it is formed of a step shaped contact end 148 in which the bottom Pumping Chamber 140b configured by the bottom case 142 has a narrow inside and a wide outside in width, and therein, the contact protruding portion 149 protruding at each contact end 148 is formed, so that the Pumping Film for preventing reverse 150 may be contacted. The Pumping Film for preventing reverse 150 compressed-fixed between the upper, bottom cases 141, 142 prevents the mother's milk from being reversed to the upper case 141 and the Pressure Generator 130 even if mother's milk is reversed through the path of extract tube for air 112 of the bottom case 142 with the structure above. In addition, the mother's milk is not remained in the bottom case 142 as the bottom case 142 is formed as a step shaped contact end 148 in which an upper portion is wider than a bottom portion in order that mother's milk would be emitted downwardly through the extract portion for air 146 at the bottom surface, even if the mother's milk is reversed.

The apparatus extracting mother's milk according to an exemplary embodiment of the present disclosure comprises:
a Hook 144 forming two or more protruding portions at the outer peripheral surface of the bottom case 142; and
a Wing portion 145 formed of a Hook hole correspondingly to the storage container 170 at the upper case 141. At this time, the combination between the upper, bottom cases 141, 142 is performed in a so-called lock and lock method that the Hook 144 of the bottom case 142 is fixed by the Hook hole of the Wing portion 145. The Hook 144 of the bottom case 142 may be formed as dented in comparison with the outer peripheral surface of the bottom case. If the Hook 144 of the bottom case 142 is formed as dented in comparison with the outer peripheral surface, efficiency of the extract action for mother's milk may be enhanced by reducing the space generated in the connector 143 of the upper, bottom cases 141, 142, when the pressure inside the upper Pumping Chamber 140a exceeds the marginal value. Hereat, the number of the Hook 144 and Hook hole may be two or more. The number of the Hook 144 and Hook hole may further be three; a skilled person in the art may understand that the number of the Hook 144 and Hook hole can be realized as an altered formation within the scope of the substance of the present disclosure.

Thus far, exemplary embodiments of the present disclosure have been explained. A skilled person in the art may understand that the present disclosure can be realized as an altered formation within the scope of the substance of the present disclosure. Therefore, the disclosed embodiments should be considered from not the limitative view point, but the explanative view point. The scope of the present disclosure is represented in not the explanations above but the following claims. It should be interpreted that all differences herein which may seem to be equal to the claims of the present disclosure are included in the present disclosure.

What is claimed is:

1. An apparatus extracting mother's milk comprising:
an Extract Container rendering extracted mother's milk to be stored with combined to an upper portion of the storage container;
an Absorber rendering mother's milk to be extracted with funneled with a path of an extract tube for mother's milk formed at a side of the Extract Container and with contacted to breast of mother;
an Extract Hose connecting the Extract Container with and a Vacuum Pump with connected with a path of an extract tube for air formed at another side of the Extract Container; and
a Vacuum Pump generating extract force,
wherein a Pumping Chamber is mounted as funneled with the path of an extract tube for air and mounted with a hose connector at the upper portion,
wherein a Pumping Film for preventing reverse blocks that mother's milk reverses to the Extract Hose with mounted in the Pumping Chamber, and renders a pumping action to perform,
and wherein a Vent Hole is formed at the Pumping Chamber and a Vacuum Removal Film is mounted at the Vent Hole.

2. A Pumping Device for the apparatus extracting mother's milk generating necessary extract force for extracting mother's milk, the Pumping Device comprising:
a Pressure Generator generating necessary pressure for extracting mother's milk;
a Pumping Chamber having upper, bottom cases formed as combined, and delivering extract force generated at the Pressure Generator to outside to be used for extracting mother's milk;
a Pumping Film for preventing reverse located in the Pumping Chamber, compressed-fixed toward the inside of the connector of the upper, bottom cases with a certain length, and rendering the Pumping Chamber divided as above and below; and
an Extract Hose delivering the pressure generated at the Pressure Generator to the Pumping Chamber,
wherein the Pumping Chamber emits partial air inside the upper Pumping Chamber to outside through the connector of the upper, bottom cases when pressure inside the upper Pumping Chamber formed by the upper case and the Pumping Film for preventing reverse exceeds marginal value.

3. The Pumping Device for the apparatus extracting mother's milk according to claim 2,
wherein the upper case is funneled by the Extract Hose connecting the Pressure Generator with the hose connector mounted at the upper portion,
wherein the bottom case includes a Rib protruding as a circle at a surface toward an inside of outer peripheral surface, and funneled with a path of an extract tube for air with formed of an extract portion for air,
and wherein the Pumping Film for preventing reverse includes a groove formed correspondingly to the Rib of the bottom case at an outer peripheral surface of the bottom portion.

4. The Pumping Device for the apparatus extracting mother's milk according to claim 2,
wherein the Pumping Film for preventing reverse has a thickness of 0.1 to 1.5 mm where the upper, bottom cases are compressed by combining.

5. The Pumping Device for the apparatus extracting mother's milk according to claim 2,
wherein the internal pressure has a marginal value of 0.1 mmHg.

6. The Pumping Device for the apparatus extracting mother's milk according to claim 2,
wherein a Hook forming two or more protruding portions is included at the outer peripheral surface of the bottom case,
and wherein a Wing portion formed of a Hook hole correspondingly to the Hook is included at the upper case.

7. The Pumping Device for the apparatus extracting mother's milk according to claim 6,
wherein the Hook of the bottom case is formed as dented in comparison with the outer peripheral surface of the bottom case.

8. The apparatus extracting mother's milk comprising:
an Extract Container rendering extracted mother's milk to be stored with combined to an upper portion of the storage container;
an Absorber rendering mother's milk to be extracted with funneled to a path of an extract tube for mother's milk formed at a side of the Extract Container and with contacted to breast of mother;
a Pressure Generator generating extract force;
a Pumping Chamber having upper, bottom cases formed as combined, and delivering extract force generated at the Pressure Generator to a path of an extract tube for air to be used for extracting mother's milk; and
a Pumping Film for preventing reverse located in the Pumping Chamber, compressed-fixed toward the inside of the connector of the upper, bottom cases with a certain length, and rendering the Pumping Chamber divided as above and below;
wherein the Pumping Chamber emits partial air inside the upper Pumping Chamber to outside through the connector of the upper, bottom cases when the pressure inside the upper Pumping Chamber formed by the upper case and the Pumping Film for preventing reverse exceeds marginal value.

9. The apparatus extracting mother's milk according to claim 8,
wherein the upper case is comprised of a hose connector at the upper portion and funneled by an Extract Hose connecting the Pressure Generator,
wherein the bottom case includes a Rib protruding as a circle at a surface toward the inside of the outer peripheral, and funneled with a path of an extract tube for air with formed of an extract portion for air,
wherein the Pumping Film for preventing reverse includes a groove formed correspondingly to the Rib of the bottom case at an outer peripheral of the bottom surface,
and wherein the Pumping Film for preventing reverse has a thickness of 0.1 to 1.5 mm where upper, bottom cases are compressed by combining.

10. The apparatus extracting mother's milk according to claim 8,
wherein the internal pressure has a marginal value of 0.1 mmHg.

11. The apparatus extracting mother's milk according to claim 8,
wherein the Pumping Chamber is mounted as integrally connected with a path of an extract tube of the Extract Container.
